(19) United States
(12) Patent Application Publication
(10) Pub. No.: US 2009/0260632 A1
(43) Pub. Date: Oct. 22, 2009

(54) Endotracheal Tube

(76) Inventors: Freddy Abnousi, San Francisco, CA (US); Celina Yong, San Francisco, CA (US)

Correspondence Address: Nixon Peabody LLP, 200 Page Mill Road, Palo Alto, CA, 94306, US

(21) Appl. No.: 12/427,629

(22) Filed: Apr. 21, 2009

Related U.S. Application Data
(60) Provisional application No. 61/047,075, filed on Apr. 22, 2008.

(51) Int. Cl. A61M 16/04

(52) U.S. Cl. 128/207.15

(57) ABSTRACT

A medical device comprises a tube to be inserted into a patient. The tube has a proximal end and a distal end and provides a first fluid into the patient's body from a fluid source via a lumen. The device includes a suction port on the tube which is configured to remove a second fluid from within the patient's body via a suction lumen. The device includes an inflatable cuff that is coupled to the outer surface of the tube. The cuff is positioned adjacent to the suction port and maintains the suction port a distance away from patient tissue. In an embodiment, the cuff is cylindrical and has an inner surface which extends from the proximal side toward the distal side, wherein the inner surface is tapered such that a diameter of the inner surface with respect to the tube decreases from the proximal side toward the distal side.

### STATEMENT OF RELATED APPLICATION(S)

The present application claims the benefit of priority based on U.S. Provisional Patent Application Ser. No. 61/047,075, filed on Apr. 22, 2008, in the name of inventor Freddy Abnousi, entitled “ENDOTRACHEAL TUBE FOR DECREASING VENTILATOR ASSOCIATED PNEUMONIA.”

### TECHNICAL FIELD

The present disclosure relates generally to medical devices, and in particular to an endotracheal tube or other appropriate tubular medical device.

### BACKGROUND

Mechanical ventilation is a staple in any modem Intensive Care Unit (ICU), and though its benefits are undeniable, it is complicated by a substantial risk of Ventilator Associated Pneumonia (VAP) occurring in the patient. With an incidence range of 9-40%, VAP is the most common nosocomial infection which occurs when the patient is in the ICU. Additionally, VAP is associated with a 15-45% attributable mortality rate and incurs significant costs to stakeholders with an additional cost ranging from $10,000-$40,000 per episode. In light of the existing clinical recommendations and devices on the market that attempt to prevent the pathogenesis of VAP, it has been recently concluded that strategies which effectively prevent VAP are urgently needed.

In the mechanically ventilated patient, though numerous factors such as critical illness, comorbidities, malnutrition, and impaired immune function compromise the patient's natural defenses. The most significant impairment which occurs to the patient is a physical result of endotracheal (ET) intubation which inhibits the patient's cough reflex, thereby impairing mucocilliary clearance and injuring the tracheal mucosa. Numerous studies have shown that ET intubation provides a direct avenue for micro-aspiration of non-sterile oropharynegeal and gastric contents into the sterile lower respiratory tract, leading to bronchopneumonia. Prevention strategies have been aimed towards decreasing aspiration into the respiratory tract and decreasing the microbial load of any possible aspiration that may occur.

A critical and independent factor in VAP prevention is aspiration prevention. FIG. 1 illustrates an existing endotracheal tube within a patient in accordance with the prior art. As shown in FIG. 1, a standard ET tube includes a balloon shaped cuff 12 near a distal portion of the tube 100. The tube 10 includes a lumen 14 built into it that allows for suction of subglottic secretions adjacent to the cuff 12. As shown in FIG. 1, tube 10 extends into the patients mouth and through the patient's throat 99 past the epiglottis 98. Reference numeral 97 represents a vocal cord of the patient and reference numeral 96 represents an area near the dorsal orifice 16 of the tube where subglottic secretions tend to pool. It has been found that use of suction orifice 16 reduces the risk of VAP by approximately 50%. However, the disadvantage of the existing system is that that the continuous suctioning via the orifice 16 produces significant tracheal injury due to the suctioning orifice coming into contact with the interior surface of the trachea and effectively pulling the tracheal tissue against the orifice via the suction force. Presently the physical barrier that exists separating the respiratory tract from the oropharynx in all intubated ICU patients is the cuff 12 of the ET tube 10. Regarding the cuff 12, it was found that the cuff 12 itself did not allow an effective seal with the interior tissue of the trachea, thereby allowing significant leakage of subglottic secretions to pass on to the patient's lungs.

### Overview

In one aspect, a medical device comprises a tube that is configured to be inserted into a patient. The tube has a proximal end and a distal end and is configured to provide a first fluid into the patient's body from a fluid source via a lumen. A suction port is configured in an outer surface of the tube and is configured to remove a second fluid from within the patient's body via a suction lumen. A cylindrical cuff is coupled to the outer surface of the tube and positioned adjacent to the suction port. The cuff is configured to be selectively inflated to a set diameter with a third fluid via a cuff lumen, the cuff having a proximal side and a distal side, wherein the cuff includes a shape having a narrowing diameter with respect to the tube from the proximal side to the distal side to direct the second fluid toward the suction port.

In an aspect, a medical device comprises a tube configured to be inserted into a patient's trachea and configured to provide a first fluid therethrough. The device includes a plurality of suction ports configured in an outer surface of the main tube, the suction ports configured to remove a second fluid from within the patient's body via a suction lumen. The device includes a plurality of cylindrical cuffs coupled to an outer surface of the tube positioned distally adjacent to a corresponding set of suction ports, the cuffs being selectively inflatable via a cuff lumen and configured to be positioned against an interior surface of the trachea when inflated to a set diameter, the cuffs each configured to direct the second fluid from within the patient's body into the respective suction ports.

In an aspect, a medical device comprises a tube to be inserted into a patient. The tube has a proximal end and a distal end and provides a first fluid into the patient's body from a fluid source via a lumen. The device includes a suction port on the tube which is configured to remove a second fluid from within the patient's body via a suction lumen. The device includes an inflatable cuff that is coupled to the outer surface of the tube. The cuff is positioned adjacent to the suction port and maintains the suction port a distance away from patient tissue. In an embodiment, the cuff is cylindrical and has an inner surface which extends from the proximal side toward the distal side, wherein the inner surface is tapered such that a diameter of the inner surface with respect to the tube decreases from the proximal side toward the distal side.

In any or all of the above, the cuff includes a first portion which extends substantially perpendicular to the outer surface of the tube. The cuff includes a second portion extending between the first portion and an interface portion, wherein the second portion is at an angle with respect to the first portion such that the second portion is configured to have a gradually decreasing diameter with respect to the outer surface from the proximal side to the distal side. Additionally or alternatively, the cuff includes at least one partially conical-shaped channel extending from the proximal side toward the distal side, the channel configured to taper to a narrower dimension toward the suction port. In an embodiment, at least one cuff includes a collection area between the proximal side and the distal side, the collection area located adjacent to a respective suction port. In an embodiment, each cuff is in communication with the common cuff lumen, although each cuff may be in communication with dedicated cuff lumens. The suction port further comprises a plurality of sets of suction ports, each set located along the tube at a respective cuff of a plurality of cuffs. The suction ports are two-way in that a fourth fluid may be applied to at least one set of suction ports via the suction lumen as well as negative pressure.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

In the drawings:

FIG. 1 illustrates an existing endotracheal tube within a patient in accordance with the prior art.

FIG. 2 is a schematic representation of the device in accordance with an embodiment.

FIG. 3A illustrates a perspective view of the device in accordance with an embodiment.

FIG. 3B illustrates a perspective view of the device in accordance with an embodiment.

FIG. 4A illustrates a cross section of the device at cuff along lines A-A in accordance with an embodiment.

FIG. 4B illustrates a cross-section of the device at cuff along lines B-B in accordance with an embodiment.

FIG. 4C illustrates a cross section of the device at cuff along lines B-B in accordance with an embodiment.

FIG. 4D illustrates a cross section of the device at cuff along lines B-B in accordance with an embodiment.

FIG. 4E illustrates a perspective view of the suction and cuff lumens of the tube in accordance with an embodiment.

FIGS. 5A-5E illustrate various designs of the cuff in accordance with one or more embodiments.

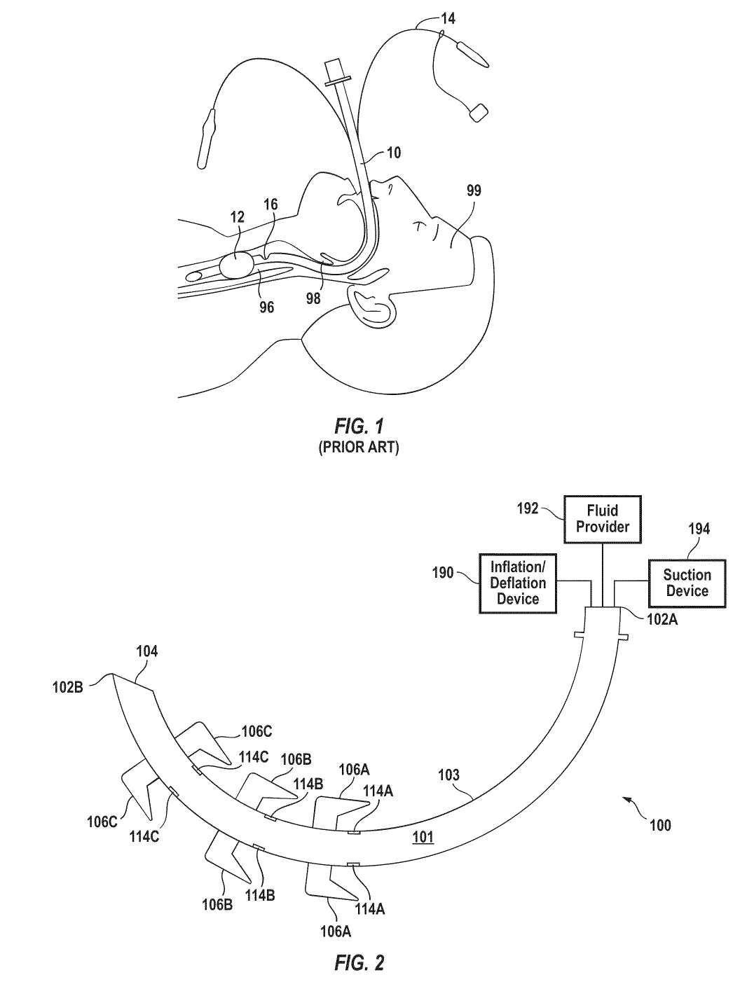

### DESCRIPTION OF EXAMPLE EMBODIMENTS

Example embodiments are described herein in the context of a medical device. Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used throughout the drawings and the following description to refer to the same or like items.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with this disclosure, the components, process steps, and/or data structures used in any type of computer or medical equipment described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. It is understood that the phrase “an embodiment” encompasses more than one embodiment and is thus not limited to only one embodiment. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Eraseable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card, paper tape and the like) and other types of program memory.

In general the endotracheal tube discussed herein is designed to prevent VAP or other injury to the patient by utilizing the inventive features described herein. In particular, the tube includes one or more specialized cuffs, one or more suction ports as well as an optional antimicrobial coating that provides for minimal microaspiration. The one or more suction ports are separated from the interior surface of the patient tissue (e.g. trachea) by the cuff or any other physical barrier such that there is no direct pressure applied by the suction port onto the interior surface of the tissue due to the presence of the physical barrier.

FIG. 2 is a schematic representation of the device in accordance with an embodiment. In particular, the device 100 is directed to a tube having a proximal end 102A and a distal end 102B. The tube 100 preferably includes a main lumen 104 which serves to provide air to the patient's lungs which is supplied by air providing device 192 (e.g. ventilator). It should be that air is only one example of fluid which is supplied to the patient through the main lumen 104 and that other types of fluids (e.g. IV, drug) are contemplated.

In an embodiment, the device 100 includes one or more selectively inflatable cuffs 106 positioned on an outer surface 104 of the tube 101, as shown in FIG. 2. It is preferred that the device 100 includes a plurality of cuffs 106 (individually, 106A, 106B, 106C) which are spaced apart from one another along the outer surface of the tube 101. The cuffs 106 are preferably operable between a deflated position and an inflated position by an inflation/deflation device 190. It is preferred that the cuffs 106 are in the deflated position when the patient is intubated, whereby the cuffs 106 are then selectively inflated once the device 100 is successfully inserted into the patient. Although it is shown in FIG. 2 that the device 100 includes three cuffs 106A, 106B, 106C on the outer surface of tube 100, more or less than three cuffs 106 are contemplated. It should also be noted that although cuffs 106 are shown in near the distal end 102B, one or more cuffs 106 may be located midway between the proximal end 102A and distal end 102B.

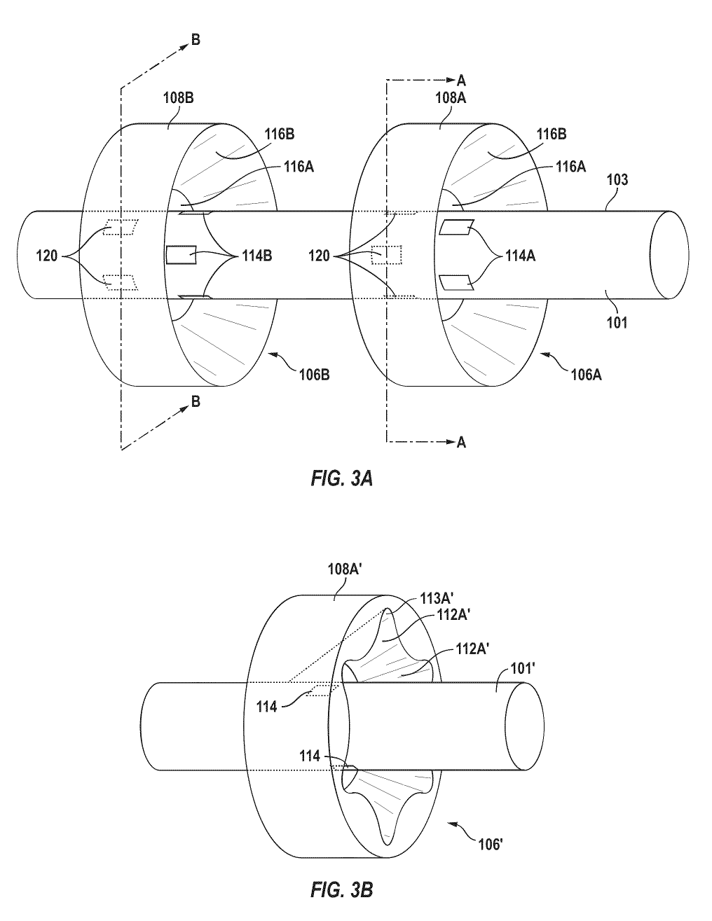

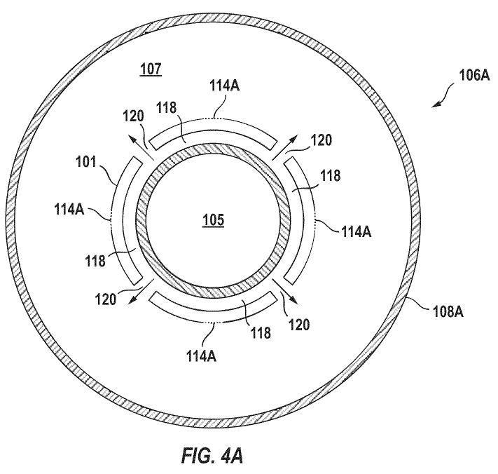

FIG. 3A illustrates a perspective view of the device in accordance with an embodiment. In an embodiment, one or more of the cuffs 106 are made of a material such as a thin, elastic plastic or other material which biologically compatible with the interior tissue of the trachea. The material will preferably allow for a superior endotracheal seal, further diminishing microaspiration risk. Of course, as stated, the device 100 may be used within other organs in which one or more of the cuffs 106 would be made of an appropriate material which is biologically compatible with the organ with which it is used. It is contemplated that one or more of the cuffs 106, suction ports, and/or tube may be lubricated with a coating which allows it to be in contact with the interior of the trachea or other passageway without causing injury to the patient. Angioplasty balloons are commonly made of PET, nylon, polymers known as cross-linked polyethylene, polyvinyl chloride, and polyurethane, all of which are plausible options for any or all components of the device 100.

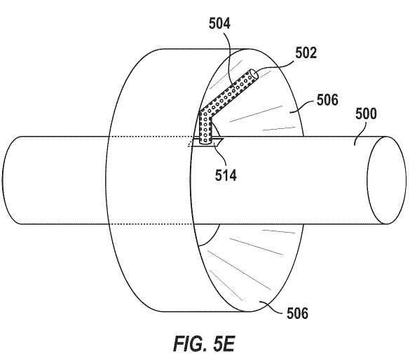

As shown in FIGS. 2 and 3, the device 100 preferably includes one or more sets of suctioning ports 114 located adjacent to and on a proximal side of the cuffs 106. In particular, as shown in FIGS. 2 and 3, a first set of ports 114A is located on the proximal side of cuff 106A, whereas a second set of suction ports 114B is located on the proximal side of cuff 106B. As shown in FIG. 2, a third set of suction ports 114C is located on the proximal side of cuff 106C. A greater or fewer number of sets of suction ports 114 are contemplated and are thus not limited to those shown in FIG. 2. It is contemplated that the suction ports 114 are not limited to being located on only the proximal side of the corresponding cuff as one or more suction ports 114 may additionally be placed on the distal side of the corresponding cuff 106 (i.e. adjacent to side 112 of the cuff 106 (see FIG. 5B). It should be noted that one or more sets of suction ports 114 may include a plurality of suction ports on the tube's outer surface 101 or as few as only one suction port 114 on the tube's outer surface 101. For sake of the brevity, although a particular set may have only one suction port, the term “suction ports” is described herein and is understood to include one or more suction ports.

The suction ports 114 are preferably apertures which are flush with the outer surface 103 of the tube, although the ports 114 may be configured to be raised with respect to the tube's outer surface 103. It is also contemplated that the ports 114 may be recessed with respect to the tube's outer surface 103 such that the portions of the tube where the suction ports 104 are located are indented with respect to the remaining portions of the tube's outer surface 103. It should be noted that the suction ports 114 may be located closer to or farther away from the interior surface of the cuff 106 from that shown in the Figures.

Additionally or alternatively, one or more suction ports may be designed to be located within the cuff itself (as shown as 514′ in FIG. 5D). In an embodiment, as shown in FIG. 5E, a suction extension member 504 in communication with the suction port 514 or directly in communication with the suction lumen extends outwards from the tube 500, whereby the suction extension member 504 removes unwanted matter. In an embodiment, the suction extension member 504 has a closed or open end 502 and includes a perforated outer surface through which vacuum is applied, thereby causing the unwanted matter to be suctioned in through the apertures in the outer surface of the member 504. It is contemplated that the member 504 be attached to the interior surface of the cuff 506 and be made of an elastic material such that the member 504 does not puncture the cuff 506 when the cuff is deflated. It should be noted that the member 504 may be used with any of the other embodiments/designs described herein.

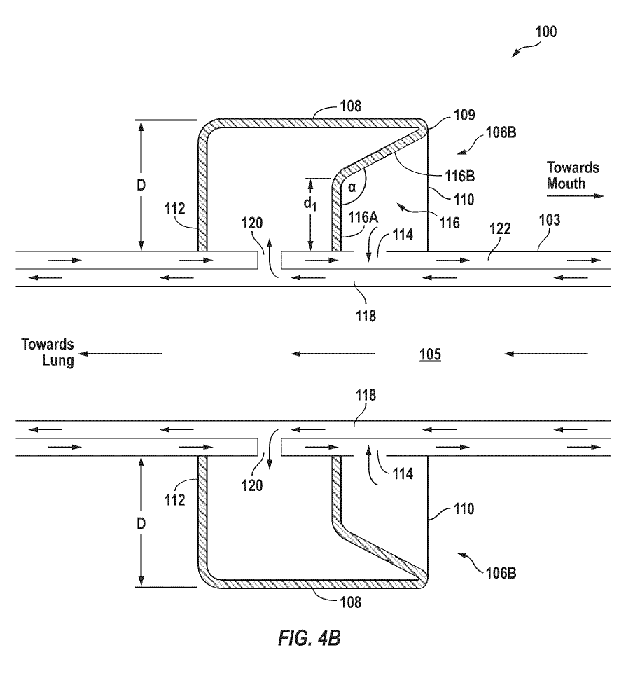

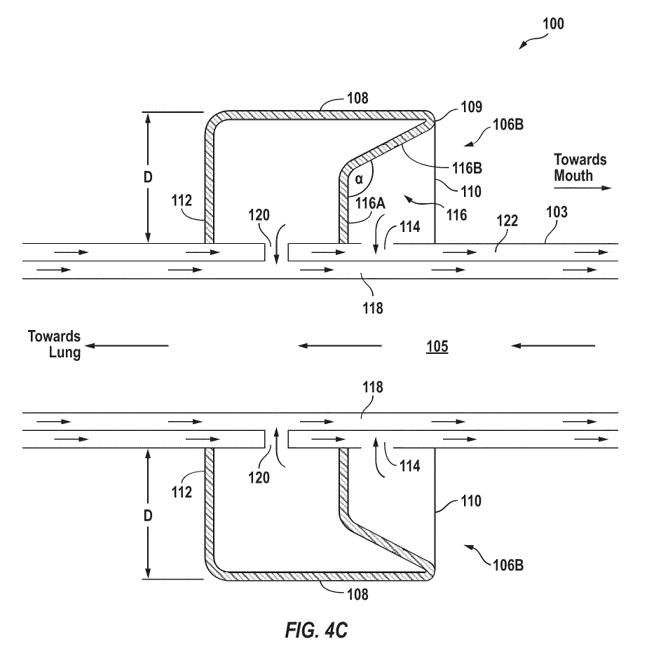

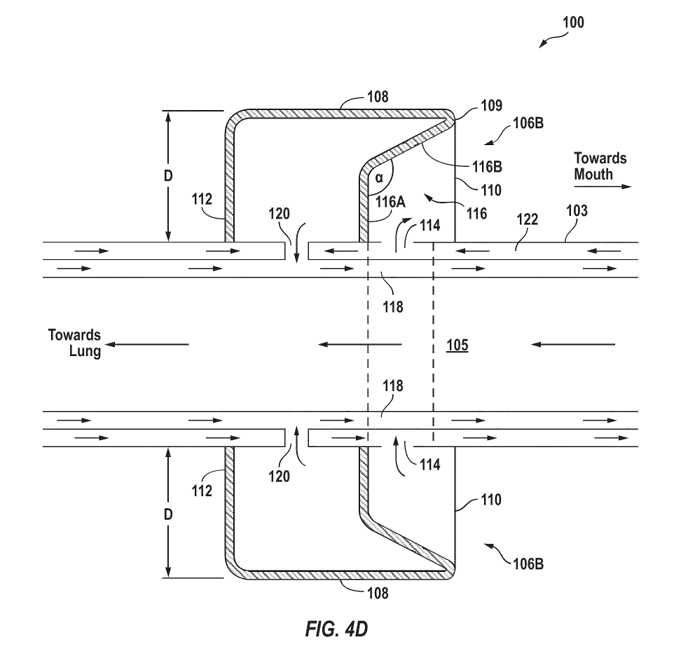

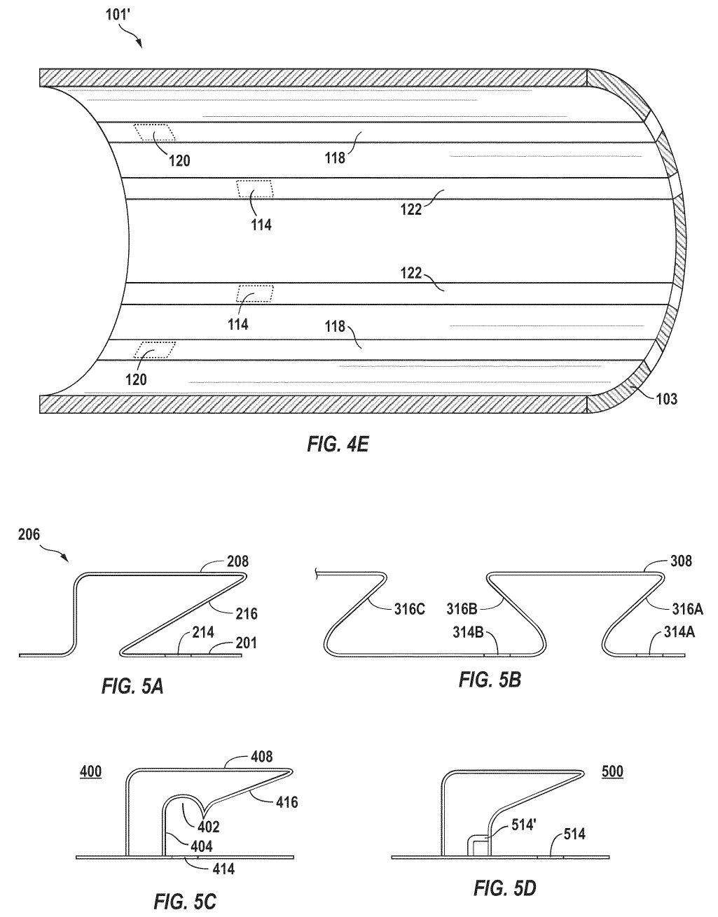

As will be discussed in more detail below, the suction ports 114 are connected to a suction lumen (FIGS. 4A-4C). It is contemplated that the device include only one suction port 114 per cuff 106, however it is preferred that a plurality of suction ports 114 are designed in the tube 101 for each cuff 106. In an embodiment, a set including a plurality of suction ports 114 may be located circumferentially in a fenestrated manner within the confines of the corresponding cuff 106, whereby the suction ports 114 are all in communication with one another via a common bridge whereby pressure applied to one lumen for that particular set is applied equally toward each of the suction ports (see phantom lines in FIG. 4D). This allows fluid to be injected via the suction lumen to one or more ports 114 whereby the fluid unclogs, dissolves and/or unseats accumulated gelatinized secretion at one or more ports 114. A vacuum may then be applied via the same suction lumen simultaneously or subsequently such that the unseated secretion is then removed from the one or more ports 114, thereby cleaning the device 100.

In an embodiment, the suctioning ports 114 are configured to remove subglottic secretions from within the trachea as the secretions pass over the ports 114. In particular, the ports 114 are coupled to one or more suction devices 194 (FIG. 2) external to the patient which apply a vacuum pressure to the ports 114 via the suction lumen (FIGS. 4A-4C). The multiple sets of suction ports 114 are preferably configured to drain the secretions and prevent the secretion from passing on to the organ of interest (e.g. patient's lungs). In an embodiment, as shown in FIG. 2, the multiple sets of suction ports 114A, 114B, 114C work in conjunction with one another to further minimize any aspirates which could potentially travel on to the patient's lungs. In particular, as described above with respect to the serially spaced cuffs 106B and 106C, the device preferably includes serially spaced suction ports 114B, 114C which function to drain and remove any secretions, fluid or other unwanted matter which may have not been otherwise collected by the proximal suction port 114A. The multiple sets of suction ports 114 thereby provide additional back up to the proximal port 114A to ensure a fail-safe prevention mechanism against infection and/or injury to the patient. It is contemplated that although the suction ports 114 are described in which a vacuum is applied to remove unwanted matter from outside the tube via the suction ports, a fluid may be applied into the suction lumen by the physician or nurse whereby the fluid is expelled through the suction ports. A vacuum may then be applied to the suction ports to removed the expelled fluid, thereby aiding in cleaning the suction ports.

The combination in the preferred device of multiple cuffs 106 as well as multiple suction ports 114 ease the burden on the nursing staff in having to decrease the clogging of the ports which would normally require manual manipulation. In particular, the device allows the nursing staff to confidently rinse the entire oropharyngeal region of the patient with substances such as chlorheaxadine or other appropriate formula or compound to decrease bacterial load without having to worry about the effect of aspiration on the patient.

One or more of the cuffs 106 preferably includes an interface surface 108 which is located between the proximal side 110 and the distal side 112 of the cuff 106 (FIG. 3B). The interface surface 108 is configured to be in sufficient contact with the interior of the trachea, when the cuff 106 is inflated to its desired amount, to prevent subglottic secretions from traveling toward (and possibly infecting) the patient's lungs.

Referring back to FIG. 2, the device 100 includes a series of cuffs 106A, 106B, 106C, one or more of which are shown to include an interface surface 108A, 108B, 108C. In the embodiment in FIG. 2, the serially spaced cuffs 106B, 106C are closer to the distal end of the device 100 than the most proximal cuff (i.e. 106A in FIG. 2) and thus provide additional barriers which prevent any subglottal secretions which escape past the proximal cuff 106A from traveling on to the patient's lungs. Thus, the subsequently located cuffs 106B, 106C serve as back-up barriers to the proximal cuff 106A such that the entire device 100 functions to provide a fail-safe mechanism in which the device 100 provides maximum protection from unwanted secretions, fluid and/or mucus from passing on to the target organ (e.g. patient's lungs), thereby preventing or inhibiting injury and/or infection to the patient (e.g. microaspiration).

In an embodiment, as shown in FIGS. 2 and 3A-3B, one or more cuffs 106 have a convergent or funnel shaped feature 116 on the proximal side 110 of the cuff 106 when the cuff 106 is in an inflated position. The convergent, funnel-like shape feature of the cuff 106 serves to direct secretions, fluids or other unwanted matter travelling downstream in the patient's trachea towards the suction ports 114 in the tube's outer surface 103.

In a particular embodiment, as shown in FIG. 4B, the feature 116 includes a bottom portion 116A which is substantially perpendicular to the outer surface 103 of the tube 101, whereby the bottom portion 116A extends vertically upward from the tube's outer surface 103 a distance d₁. In the embodiment shown in FIG. 4B, the feature 116 includes a top portion 116B which is oriented at an angle a with respect to the bottom portion 116A whereby the top portion 116A gradually extends upward with respect to the tube's outer surface 103 toward the interface surface 108. This tapered configuration 116 gradually directs secretions and/or other unwanted matter in a natural flow-like manner toward the suction ports 114. In addition, it is preferred that the top portion 116A meets the interface surface 108 at junction 109 to form a sharp or rounded corner between the proximal side 110 and the interface side 108 of the cuff 106 to further provide a robust seal between the cuff 106 and the interior surface of the trachea. In addition, the cuff 106 maintains the suction port 114 a predetermined distance (i.e. distance D) from the interior wall of the trachea, thereby preventing the suction port 114 from coming into contact and damaging the interior of the trachea. It is preferred that the suction port 114 is located in the tube at a location in which it is covered by the cuff 106. In other words, the suction port 114 is preferably not exposed laterally along the tube 101 outside the width dimension of the cuff 106.

Thus, the configuration of the cuff 106 along its proximal side serves multiple purposes in not only preventing unwanted matter from travelling past the cuff 106 (via the interface surface) but also gradually directing the unwanted matter toward the suction ports 114 and preventing the suction port 114 from coming into contact with the interior surface of the trachea. This not only decreases pooling of microaspirations in a supine patient but also allows for directed suctioning that does not affect the tracheal mucosa.

It should be noted that the above described cuff 106 is only an example and may exhibit other shapes and configurations which perform substantially the functions of directing the unwanted matter toward the suction ports 114 and preventing the matter from travelling downstream past the cuff 106. For instance, in an embodiment shown in FIG. 3B, the cuff 106′ may have a ruffled configuration such that portions of the interior surface have a wider surface area than other portions of the cuff 106′ to form channels 112A′. These channels 112A′ are configured to direct the secretion material toward suction ports 114 which are located at the base of one or more of the channels 112A′. Each channel 112A′ is preferably configured to have a respective conical shape which tapers vertically and concentrically from the proximal edge of the cuff 113A′ toward the point where the cuff comes into contact with the outer surface 101′ of the tube. It is contemplated in this example that the cuff 106′ thereby have a plurality of individual conical channels which extend from the proximal side of the cuff 106′ toward the point between where the cuff 106′ and outer tube 101′ meet.

In another embodiment, as shown in FIG. 5A, the cuff 206 may be configured to have only the angled portion 216 which extends from the interface surface 208 to the tube's outer surface 201 such that the cuff 206 does not incorporate the bottom portion described above. In an embodiment, as shown in FIG. 5B, the cuff 306 includes funnel shaped configurations on the distal side 316B as well as the proximal side 316A. Suction ports 314A and 314B may be configured on the corresponding proximal and distal sides 316A, 316B as shown in FIG. 5B. Although not shown in FIG. 5B, a suction port may be configured next to surface 316C of the cuff subsequent to cuff 308.

It is additionally/alternatively contemplated, as shown in FIG. 5C, that the cuff 400 has the upper portion 416 which vertically tapers toward the tube, whereby the cuff 400 includes a cutaway area 402 directly above the suction port 414, whereby secretions are pooled in the cutaway area 402 prior to be removed via the suction port 414. It should be noted that any or all of the above cuff designs may be incorporated alone or in combination with any or all of the other cuff designs within the scope of the device. For example, the cuff may include conical channels in FIG. 3B combined with the cutaway areas to achieve maximized collection and removal of unwanted matter.

In the embodiment shown in FIG. 2, the cuffs 106A, 106B, 106C are preferably sequentially separated by a predetermined distance along the length of the tube and can be inflated and deflated individually and/or together by applying a desired amount fluid inside the cuffs 106. In particular, the cuffs 106 are in communication with one or more corresponding cuff lumens, whereby fluid applied through the cuff lumen may be selectively directed to or away from one or more selected cuffs 106 to inflate or deflate the cuffs 106.

FIG. 4A illustrates a cross section of the device 100 at cuff 106A along lines A-A in accordance with an embodiment. FIG. 4B illustrates a cross-section of the device 100 at cuff 106B along lines B-B in accordance with an embodiment. In particular to FIG. 4B, the device 100 as shown includes a suction lumen 122 in communication with the suction port 114B, whereby a vacuum is preferably applied to the suction port 114B via suction lumen 122. The vacuum may be applied continuously either manually or automatically by a person or a machine. It is also contemplated that the vacuum may be applied intermittently manually or automatically by a person or a machine. For example, the suction device 194 in FIG. 2 may run on software which applies a vacuum of one or more of the suction ports 114 at scheduled time without the need for a physician or nurse to manually apply the vacuum.

The suction lumen 122 is integrated into the tube 101, whereby the lumen 122 is shown and integrated in the tube 101 and immediately below the tube's outer surface 103. However, it is contemplated that the lumen 122 may be alternatively located elsewhere inside the tube 101 or outside of the tube 101. For instance, the lumen 122 may be a separate tube positioned within the lumen cavity between the outer tube 103 and the inner lumen 105. In an embodiment, all suction ports 114A, 114B, 114C share a common suction lumen 122. In another embodiment, each suction port 114 is in communication with a dedicated suction lumen 122, whereby vacuum pressure in a particular suction lumen 122 will not affect pressure fluid in another suction lumen 122. This allows selective and independent control of the amount of suction pressure applied to each suction port 114. It is contemplated that fluid be injected into the suction lumen 122 (i.e. opposite to the arrows shown in FIGS. 4A and 4B), whereby fluid may be used to rinse and clear one or more suction ports 114 of accumulated secretions. In an example, as shown in FIG. 4D, rising fluid such as chlorhexidine or the like may be injected into suction lumen 122 whereby the rinsing fluid applied to one or more affected suction ports. In that example, vacuum may be applied to another suction lumen 122 whereby the injected fluid is almost immediately removed from another (or the same) suction port. This allows the device 100 to unclog one or more suction ports of solidified secretions and/or mucus such that the device 100 continues to effectively operate. In the example above, it is contemplated that rinsing fluid may be applied only at a directed time to one or more of the suction lumens 122, whereby vacuum pressure is not applied to any of the suction lumens 122 until the rinsing process has terminated.

As shown in FIGS. 4A and 4B, a cuff lumen 118 preferably within the tube 101 is in communication with the interior of the cuff 106 via one or more ports 120, whereby fluid applied from the inflation/deflation device 190 or other appropriate equipment enters the cuff 106 via the one or more ports 120 to inflate the cuff 106. In contrast, the cuff 106 may be deflated in which fluid within the cuff 106 would be removed via the cuff lumen 118 back toward the inflation/deflation device 190, as shown by the arrows in FIG. 4C. It is contemplated that the cuffs 106A, 106B, 106C may be serially connected via appropriate valve technology such that one cuff lumen 118 is used to individually and/or collectively inflate or deflate one or more cuffs 106 in accordance with an embodiment. It is contemplated as well that the cuffs 106A, 106B, 106C may each have a dedicated cuff lumen which operates only the corresponding cuff to allow any of the cuffs to be selectively operated at will.

It should be noted that although the cuff lumen 118 is shown positioned below the suction lumen 112 in FIG. 4B, the cuff lumen 118 may alternatively be located immediately below the outer surface 103 of tube 101 and thus above the suction lumen 122. In an embodiment, as shown in FIG. 4D, the suction lumens 122 and cuff lumens 118 may be integrated into the tube 101, whereby the lumens 118, 112 are adjacent to and concentric with respect to a center of the tube 101.

In an embodiment, the cuffs 106 share a common cuff lumen 118 whereby fluid provided via the lumen 118 will inflate all of the cuffs 106. In another embodiment, each cuff 106 has ports 120 which are in communication with a dedicated cuff lumen 118, whereby fluid injected or removed from that particular cuff lumen 118 will not affect fluid in another cuff lumen 118 which is connected to another cuff 106. This allows inflation/deflation of each cuff 106 to be independently controlled by the physician or nurse.

In an embodiment, the fluid applied to one or more of the cuffs 106 is air, however this is not limited thereto. For example, other fluids that are denser or lighter than air may be applied to the cuffs 106. It is also contemplated that a mixture of different fluids may be applied to one or more of the cuffs 106 to inflate the cuffs 106. For example, it is contemplated that 3-4 cc of a gel or other compound denser than air be applied to one or more of the cuffs 106 which is then followed by an injection of 1-2 cc of air and/or vice versa. The use of a dense silica gel in inflating the cuff 106 allows the cuff 106 to sufficiently mold to the interior surface of the patient's trachea while maintaining low pressures and avoiding tracheal collateral damage. Additionally, the use of a dense silica gel for inflation, followed by minimal air, allows for maintenance of cuff pressures for a significantly longer period of time, easing the work of nursing staff, and allowing cost savings in not having to purchase an external device to monitor and maintain cuff air pressures. It should be noted that other measurements of fluid besides those indicated above are contemplated for use with the device 100.

It is contemplated that the device 100 may be configured to incorporate one or more integrated sensors within the cuff or other location in the device which monitors any leakage from the cuffs 106 provides such relevant data to the physician or nurse. It is also contemplated that the device be configured to operate in conjunction with one or more sensors external to the patient, such as in the fluid providing external device (e.g. backpressure sensor) which alerts the physician or nurse of fluid leakage from within the cuff 106. Such leakage information may be used to compensate for leakage by applying more fluid into the one or more cuffs 108.

It is contemplated that pressure to the one or more cuffs and/or suction ports can be dynamically adjusted to minimize or avoid damage to the trachea when the device 100 is kept within the patient for long term intubation. In an example, one cuff (for instance cuff 106A) may be deflated a desired amount to minimize damage to that portion of the trachea while one or more of the remaining cuffs are inflated further or maintained at their inflated position to prevent unwanted matter from passing on to the organ of interest. The adjustment feature of the device may avoid the need for a tracheotomy since the device could be adjusted to prevent damage to the patient's trachea.

It is contemplated that at least a portion of the tube be made of material which incorporates Silver or its alloys, Copper or its alloys, or a combination thereof. It has been found that the combination of Silver and Copper have shown to have greater antimicrobial activity than a single metal. Additionally, the combination of these two metals have found to decrease biofilm formation and bacterial load as well as cost effectiveness for manufacturing purposes. It should be noted that the above materials are only preferred and any other appropriate materials or combinations thereof may be used. It is contemplated that the suction ports, cuffs and/or tube may be configured to have hydrophobic, hydrophilic, lipophobic or lipophillic properties to ensure that the secretions do not pass onto the organ of interest and are properly removed by the device 100. It is envisioned that the cuffs, suction ports, and/or tube is coated with an antimicrobial and/or antibiofilm coating, such as chlorohexadine, eluting and eluting surfactants, eluting and non-eluting antibiotics, Heparin or the like to prevent formation of biofilms or other accumulated unwanted matter on the device 100. It is also contemplated that the device 100 be coupled to a high frequency signal generator which applies a high radio frequency (RF) signal to electrodes configured on the outer surface or any other part of the tube and/or other components of the device 100 to kill harmful bacteria. It is contemplated that the device be used with a germicidal light source to kill harmful bacteria.

It should be noted that although the device is described above in relation to preventing subglottic secretions from passing to the patient's lungs, it is contemplated that the device may be used in other medical applications in which the device is used to prevent other undesired fluids, bacteria, fungus and/or viruses from traveling downstream or upstream to a restricted site or organ within the patient. For instance, the device may be designed to be used prevent bacteria or fungi from traveling to the patient's urinary tract during an operation.

Other organs this device may be used include within the venous system as a method for filtering or blocking clots, pulmonary vessels as a way for minimizing bacterial spread or clot progression, coronary vessels for blocking clots or atherosclerotic plaques from advancing to undesirable distal regions. The device may be designed to be used in a gastrointestinal (GI) surgery/procedure to repair esophageal bleeding, bariatric surgery, or any other GI tract surgery that would require control of tissue and/or prevention of flow of fluids. The device may be designed to be used in cardiovascular surgery or procedures in which the device would be used in the prevention of flow, stabilization of tissue, retrieval of clots. The device may be designed to be used in thoracic procedures such as bronchial repair, selective lung inflation/deflation in cardio thoracic surgery. It should be noted that the dimensions of the tube and/or cuff may be changed as well as the ratios between the tube and the cuff may be changed to use the device for specific procedures or patients.

While embodiments and applications have been shown and described, it would be apparent to those skilled in the art having the benefit of this disclosure that many more modifications than mentioned above are possible without departing from the inventive concepts disclosed herein. The invention, therefore, is not to be restricted except in the spirit of the appended claims.

What is claimed is:

1. A medical device comprising:
a tube configured to be inserted into a patient, the tube having a proximal end and a distal end, the tube configured to provide a first fluid into the patient's body from a fluid source via a lumen;
a suction port configured in an outer surface of the tube, the suction port configured to remove a second fluid from within the patient's body via a suction lumen within the tube; and
a cylindrical cuff coupled to the outer surface of the tube and positioned adjacent to the suction port, the cuff configured to be selectively inflated to a set diameter with a third fluid via a cuff lumen, the cuff having a proximal side and a distal side, wherein the cuff includes a funnel shape of narrowing diameter from the proximal side to the distal side to direct the second fluid toward the suction port.

2. The device of claim 1, wherein the cuff further comprises a plurality of cuffs coupled to the tube and separated by a distance along a length of the tube.

3. The device of claim 2, wherein each cuff is in communication with the cuff lumen.

4. The device of claim 2, wherein at least one cuff in the plurality is in communication with a dedicated cuff lumen separate from the remaining cuffs in the plurality.

5. The device of claim 1, wherein the cuff includes a first portion extending substantially perpendicular to the outer surface of the tube, the cuff including a second portion extending between the first portion and an interface portion, wherein the second portion is at an angle with respect to the first portion such that the second portion is configured to have a gradually decreasing diameter with respect to the outer surface from the proximal side to the distal side.

6. The device of claim 1, wherein the cuff includes at least one partially conical-shaped channel extending from the proximal side toward the distal side, the channel configured to taper to a narrower dimension toward the suction port.

7. The device of claim 1, wherein the suction port further comprises a plurality of sets of suction ports, each set located along the tube at a respective cuff of a plurality of cuffs.

8. The device of claim 1, wherein at least one cuff includes a collection area between the proximal side and the distal side, the collection area located adjacent to a respective suction port.

9. The device of claim 7, wherein a fourth fluid is applied to at least one set of suction ports via the suction lumen.

10. The device of claim 9, wherein a negative pressure is applied to the at least one set of suction ports to remove the fourth fluid.

11. A medical device comprising:
a tube configured to be inserted into a patient's trachea and configured to provide a first fluid therethrough;
a plurality of suction ports configured in an outer surface of the main tube, the suction ports configured to remove a second fluid from within the patient's body via a suction lumen; and
a plurality of cylindrical cuffs coupled to an outer surface of the tube positioned distally adjacent to a corresponding set of suction ports, the cuffs being selectively inflatable via a cuff lumen and configured to be positioned against an interior surface of the trachea when inflated to a set diameter, the cuffs each configured to direct the second fluid from within the patient's body into the respective suction ports.

12. The device of claim 11, wherein each cuff is in communication with the cuff lumen.

13. The device of claim 11, wherein at least one cuff in the plurality is in communication with a dedicated the cuff lumen separate from the remaining cuffs in the plurality.

14. The device of claim 11, wherein at least one cuff includes a first portion extending substantially perpendicular with respect to the outer surface of the tube, the cuff including a second portion extending between the first portion and an interface portion, wherein the second portion is at an angle with respect to the first portion such that the second portion is configured to have a gradually decreasing diameter with respect to the outer surface from a proximal side to a distal side of the cuff.

15. The device of claim 11, wherein at least one cuff includes at least one conical channel extending from a proximal surface of the cuff, the channel configured to narrowly taper toward the suction port.

16. The device of claim 11, wherein at least one cuff includes a collection area between the proximal side and the distal side, the collection area located adjacent to a respective suction port.

17. The device of claim 11, wherein a fourth fluid is applied to at least one set of suction ports.

18. The device of claim 17, wherein a negative pressure is applied to at least one set of suction ports to remove the fourth fluid.

19. A medical device comprising:
a tube configured to be inserted into a trachea of a patient, the tube having a proximal end and a distal end, the tube configured to provide a first fluid into the patient's body from a fluid source via a lumen;
a suction port configured in an outer surface of the tube, the suction port configured to remove a second fluid from within the patient's body via a suction lumen; and
an inflatable cuff coupled to the outer surface of the tube and positioned adjacent to the suction port, the cuff configured to maintain the suction port a distance away from an interior surface of the trachea.

20. The device of claim 19 wherein the cuff is cylindrical in shape and has a proximal side and a distal side, the cuff including an inner surface extending from the proximal side toward the distal side, the inner surface being tapered such that a diameter of the inner surface with respect to the tube decreases from the proximal side toward the distal side.
